(19) United States
(12) Patent Application Publication
(10) Pub. No.: US 2015/0150637 A1
(43) Pub. Date: Jun. 4, 2015

(54) MEDICAL MANIPULATOR, MEDICAL SYSTEM INCLUDING THE SAME, MEDICAL PUNCTURING SYSTEM, AND BIOPSY SYSTEM

(71) Applicant: CANON KABUSHIKI KAISHA, Tokyo (JP)

(72) Inventor: Takeshi Iwasa, Tokyo (JP)

(21) Appl. No.: 14/407,229

(22) PCT Filed: Jun. 4, 2013
(86) PCT No.: PCT/JP2013/065907

§ 371 Date: Dec. 11, 2014

(30) Foreign Application Priority Data
Jun. 15, 2012 (JP) 2012-135452

(51) Int. Cl. A61B 19/00; A61B 10/04; A61B 17/34

(52) U.S. Cl. CPC A61B 19/2203; A61B 17/3478; A61B 10/04; A61B 19/5244; A61B 19/201; A61B 2019/2253; A61B 2017/00539

(57) ABSTRACT

The present invention relates to a medical manipulator equipped with a fail-safe mechanism capable of protecting a manipulator in an emergency. A medical manipulator includes a supporting section; a driving section supported by the supporting section and including a vibrating body in which vibration waves are excited by application of an alternating voltage, a movable body that moves relative to the vibrating body by receiving the vibration waves, and a pressurization unit configured to exert pressure between the vibrating body and the movable body; a manipulator section supported by the driving section; an emergency detecting unit configured to detect an emergency; and an interruption unit configured to interrupt transmission of torque between the manipulator section and the supporting section on the basis of an emergency detection signal transmitted from the emergency detecting unit.

### TECHNICAL FIELD

The present invention relates to a medical system including a vibration-type actuator as a driving source, and in particular, it relates to a fail-safe mechanism for protecting a medical manipulator including a vibration-type actuator as a driving source.

### BACKGROUND ART

Medical robotic systems, such as a manipulator, have been studied actively in recent years. A biopsy system using a magnetic resonance imaging (MRI) system is a good example, in which the user controls the position of a manipulator while viewing an MR image to perform a biopsy at a high-accuracy collecting position. The MRI gives the measurement site of a subject a static magnetic field and a specific high-frequency magnetic field, thereby imaging it by applying a nuclear magnetic resonance phenomenon generated in the body of the subject.

Since MRI uses a high magnetic field, a general electromagnetic motor cannot be used as a power source for the manipulator. Therefore, various systems that use a vibration-type actuator represented by an ultrasonic motor as a power source have been proposed. For example, PTL 1 discloses a method for visually stimulating the subject under a high magnetic field environment by using a method referred to as functional MRI.

Furthermore, NPL 1 discloses paracentesis and a puncturing device system using MRI.

### CITATION LIST

### Patent Literature

PTL 1 Japanese Patent Laid-Open No. 2011-245202

### Non Patent Literature

NPL 1 “Compact manipulator system for guiding needle with real-time navigation based on MR images”, Journal of Japan Society of Computer Aided Surgery 9(2): 91-101, 2007

### SUMMARY OF INVENTION

### Technical Problem

A vibration-type actuator can adopt direct drive, in which no reduction gear is needed, for an electromagnetic motor that uses Lorentz force as a driving force because of its feature of low-speed high-torque operation. Accordingly, the vibration-type actuator has an advantage in that high-accuracy, high-response control with reduced backlash, so that application to a medical manipulator is expected. However, there is the following problem in applying the vibration-type actuator to a medical manipulator as a power source.

An electromagnetic motor that uses Lorentz force as a driving force has a structure in which the rotor and the stator are not in contact with each other. Because of the structure, such an electromagnetic motor generates no torque, except a cogging torque and a friction torque due to a reduction gear, while not energized. In contrast, the vibration-type actuator has the characteristic that it has torque between a stator serving as a vibrating body and a rotor serving as a movable body, even if not energized, from the characteristic that a motive force is transmitted from the vibrating body to the movable body due to the frictional force therebetween. Accordingly, assuming an emergency, for example, a case where external power supply to the medical manipulator is shut off while the medical manipulator is in operation, a medical manipulator including a vibration-type actuator as a power source, which is in contact with the subject, may remain in the contact state. This causes a problem in that the manipulator is damaged due to the motion of the subject.

An application of the medical manipulator is a medical system in which the manipulator is combined with a medical imaging apparatus, as disclosed in PTL 1. In the original operation of such a medical system, the amount of manipulation of the manipulator can be controlled while the positional relationship between a site of the subject and the manipulator is quantitatively ascertained, which offers an advantage in that a higher-accuracy remote manipulation can be achieved. On the other hand, it can also be said that such a medical system operates on the precondition that it is under combined control of a manipulating system, an image observation system, a monitoring system, and so on. Accordingly, this indicates that, assuming an emergency, such as shut-off of power supply to such a medical system, a decrease in the control performance of the medical system occurs in the manipulating system, the image observation system, and the monitoring system at the same time. Accordingly, a medical manipulator that may be used in combination with a medical imaging apparatus requires a fail-safe mechanism for protecting the manipulator more reliably.

### Solution to Problem

The present invention provides a medical manipulator including a supporting section; a driving section supported by the supporting section and including a vibrating body in which vibration waves are excited by application of an alternating voltage, a movable body that moves relative to the vibrating body by receiving the vibration waves, and a pressurization unit configured to exert pressure between the vibrating body and the movable body; a manipulator section supported by the driving section; an emergency detecting unit configured to detect an emergency; and an interruption unit configured to interrupt transmission of torque between the manipulator section and the supporting section on the basis of an emergency detection signal transmitted from the emergency detecting unit.

### Advantageous Effects of Invention

As described above, according to some embodiments of the present application, a medical manipulator equipped with a vibration-type actuator can be provided with a fail-safe mechanism that prevents a supporting section and a manipulator section of the medical manipulator from being locked in an emergency, such as a power failure.

Furthermore, according to some embodiments of the present application, a medical manipulator equipped with a vibration-type actuator can be provided with a fail-safe mechanism that prevents an influence of a static torque between a vibrating body (stator side) and a movable body (rotor side) of the vibration-type actuator in an emergency, such as a power failure.

Furthermore, according to some embodiments of the present application, a vibration-type actuator applied to a driving source of a medical system equipped with a fail-safe mechanism that interrupts the static torque itself of a vibrating body (stator side) and a movable body (rotor side) can be provided.

Further features of the present invention will become apparent from the following description of exemplary embodiments with reference to the attached drawings.

### BRIEF DESCRIPTION OF DRAWINGS

FIG. 1A is a block diagram showing a connection configuration of a medical manipulator according to a first aspect of the present invention.

FIG. 1B is a block diagram showing a connection configuration of the medical manipulator according to the first aspect of the present invention.

FIG. 1C is a block diagram showing a connection configuration of the medical manipulator according to the first aspect of the present invention.

FIG. 1D is a block diagram showing a connection configuration of the medical manipulator according to the first aspect of the present invention.

FIG. 1E is a block diagram showing a connection configuration of the medical manipulator according to the first aspect of the present invention.

FIG. 1F is a block diagram showing a connection configuration of a driving section in the medical manipulator according to the first aspect of the present invention.

FIG. 1G is a block diagram showing a connection configuration of the driving section in the medical manipulator according to the first aspect of the present invention.

FIG. 2A is a block diagram showing a connection configuration of a medical manipulator according to a second aspect of the present invention.

FIG. 2B is a block diagram showing a connection configuration of the medical manipulator according to the second aspect of the present invention.

FIG. 2C is a block diagram showing a connection configuration of the medical manipulator according to the second aspect of the present invention.

FIG. 2D is a block diagram showing a connection configuration of the medical manipulator according to the second aspect of the present invention.

FIG. 3A is a diagram showing a configuration in which a medical manipulator according to an embodiment of the present invention is applied to a medical puncturing system.

FIG. 3B is a diagram showing a configuration in which a medical manipulator according to an embodiment of the present invention is applied to a medical biopsy system.

FIG. 4 is a block diagram showing the connection relationship between a medical manipulator and a driving unit according to an embodiment of the present invention.

FIG. 5 is a block diagram showing the connection relationship between a medical manipulator of an embodiment of the present invention and an external unit.

FIG. 6A is a schematic cross-sectional view of an embodiment according to a second aspect of the present invention.

FIG. 6B is a schematic cross-sectional view of an embodiment according to the second aspect of the present invention.

FIG. 7A is a diagram illustrating the positional relationship between a medical manipulator according to an embodiment of the present invention and a subject.

FIG. 7B is a diagram illustrating the positional relationship between the medical manipulator and the subject.

FIG. 7C is a diagram illustrating a connection configuration of the medical manipulator according to the embodiment of the present invention and a medical imaging apparatus.

FIG. 7D is a diagram illustrating the connection configuration of the medical manipulator and the medical imaging apparatus.

FIG. 8A is a block diagram showing the connection configuration of a conventional medical manipulator.

FIG. 8B is a block diagram showing a connection configuration of a driving section of the conventional medical manipulator.

FIG. 8C is a block diagram showing a connection configuration of the driving section of the conventional medical manipulator.

FIG. 9 is a schematic cross-sectional view showing a configuration example of a vibration-type actuator applicable to the present invention.

FIG. 10A is a schematic diagram of a mechanical clutch applicable to the first aspect of the present invention.

FIG. 10B is a schematic diagram of an example of the mechanical clutch applied to a biopsy system.

FIG. 10C is a schematic diagram of an example of the mechanical clutch applied to a biopsy system.

### DESCRIPTION OF EMBODIMENTS

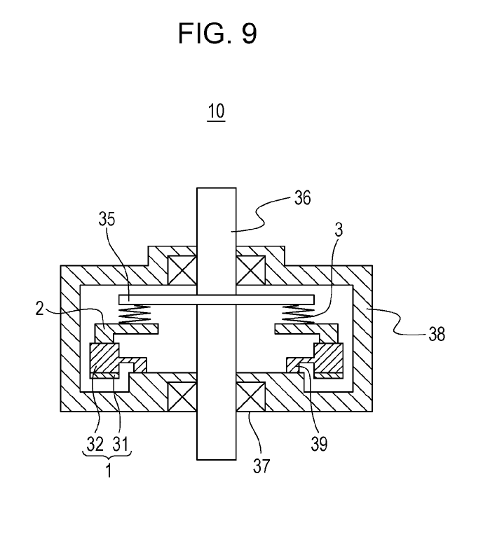

Referring first to FIG. 9, a vibration-type actuator applicable to a medical manipulator according to an embodiment of the present invention will be described. FIG. 9 is a schematic cross-sectional view showing, in outline, the configuration of a ring-shaped ultrasonic motor 10. In FIG. 9, an interruption unit 5, which is one of the characteristics of a first aspect and a second aspect of the present invention, and a pressure control unit 7, which is one of the characteristics of the second aspect of the present invention, are omitted for simplicity.

A piezoelectric device 31 is bonded in a ring shape to a ring-shaped vibrator 32. The piezoelectric device 31 excites vibration in response to an electrical signal applied. The electrical signal applied to the piezoelectric device 31 includes at least AC voltage. The vibrator 32 amplifies the vibration excited by the piezoelectric device 31 as flexural vibration. A pressure applied by a pressurization unit 3 is exerted between a movable body 2 and the vibrator 32. The vibration of the vibrator 32 is transmitted to the movable body 2 due to a frictional force, so that the movable body 2 rotates. The movable body 2 and a torque transmission member 35 are connected by the pressurization unit 3, so that the rotation of the movable body 2 rotates an output shaft 36. The output shaft 36 is configured to rotate relative to a housing 38 by using a bearing 37. On the other hand, the vibrator 32 is fixed to the housing 38 by using a joining unit 39. In the present invention, a structure formed of the vibrator 32 and the piezoelectric device 31 is referred to as a vibrating body 1.

Although a ring-shaped type in which the vibrating body 1 and the movable body 2 are disposed in a ring shape, with the rotation shaft 36 as the center, is shown in FIG. 9 as the vibration-type actuator applicable to the medical manipulator according to an embodiment of the present invention, the present invention is not limited to the ring-shaped type. For example, the present invention includes a linear type in which the vibrating body 1 and the movable body 2 are disposed in a line and a tubular type in which one of the vibrating body 1 and the movable body 2 and the other are disposed in an inner tube and an outer tube that constitute a double tube, respectively.

The pressurization unit 3 is configured to exert pressure in the axial direction of the rotation shaft 36 but not to be deformed in the rotating direction and may be a disc spring or the like. The vibration-type actuator has a static torque due to the axial pressure of the pressurization unit 3, which is a considerably different characteristic from that of an electromagnetic motor that uses Lorentz force as a driving force. The vibration-type actuator has the advantage of being capable of a low-speed, high-torque operation as compared with electromagnetic motors, and thus has the characteristic that a direct drive mechanism without a reduction gear can easily be adopted. Also in the present invention, the vibration-type actuator may adopt the direct drive mechanism.

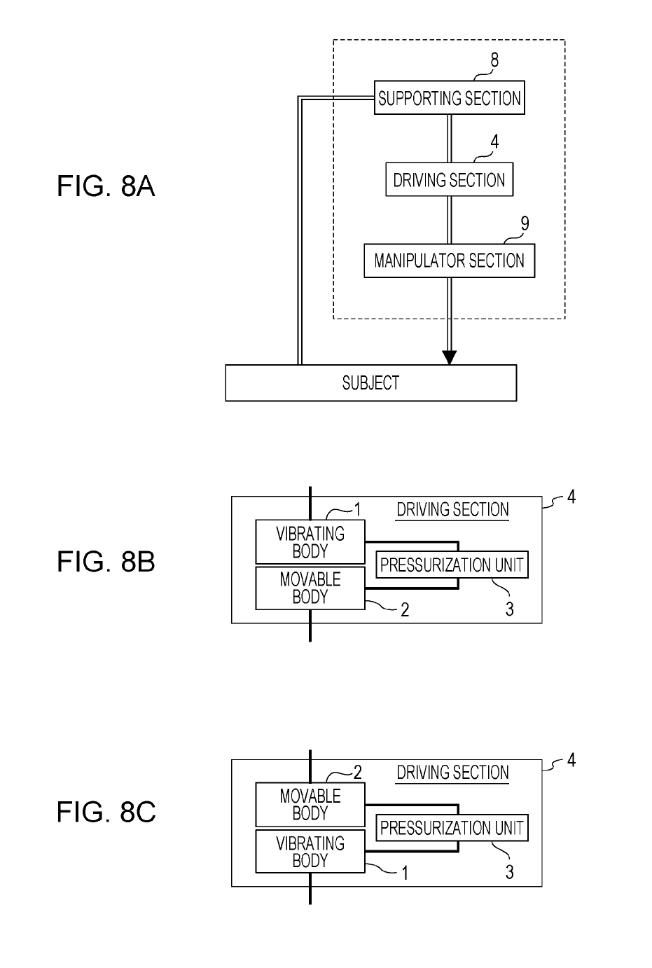

Referring next to FIGS. 8A to 8C, an example of the configuration of a conventional manipulator and problems thereof will be described. FIG. 8A shows the connection relationship of a manipulator including a driving section 4 shown in FIG. 8B or 8C.

The conventional manipulator shown in FIG. 8A does not include the interruption unit 5, an emergency detecting unit 6, and the pressure control unit 7, which are features of the present invention. Accordingly, the interval between the vibrating body 1 and the movable body 2 is constantly pressurized by the pressurization unit 3, so that a frictional force is generated irrespective of the driving state of the driving section 4, that is, the vibration-type actuator. Furthermore, there is no disconnection mechanism that mechanically disconnects the connection among a supporting section 8, the driving section 4, and a manipulator section 9 on the path from the supporting section 8 through the driving section 4 to the manipulator section 9. Accordingly, a static torque is generated between the supporting section 8 and the manipulator section 9 irrespective of the driving state of the driving section 4. This may cause the end of the manipulator section 9 to be fixed to the subject in an emergency due to power failure or the like.

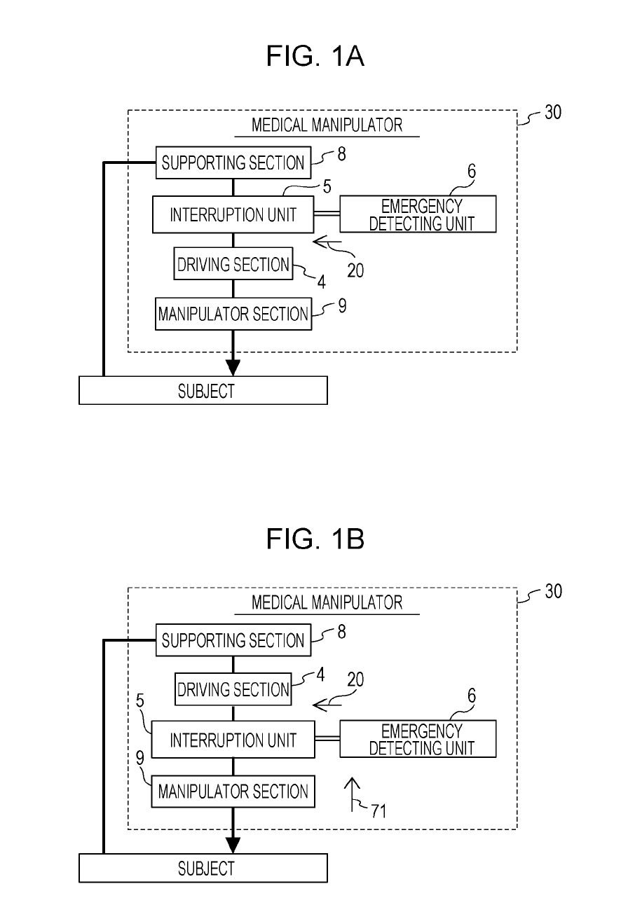

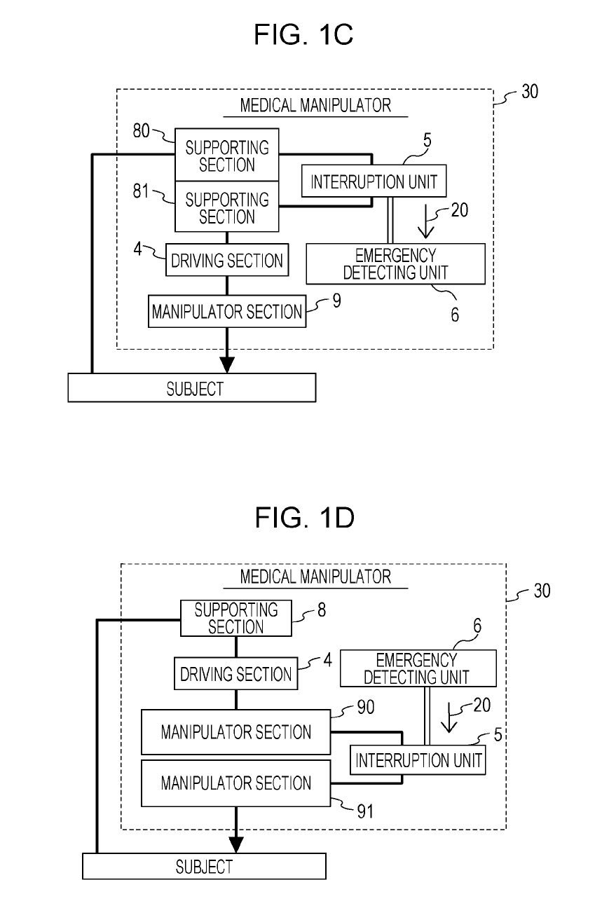

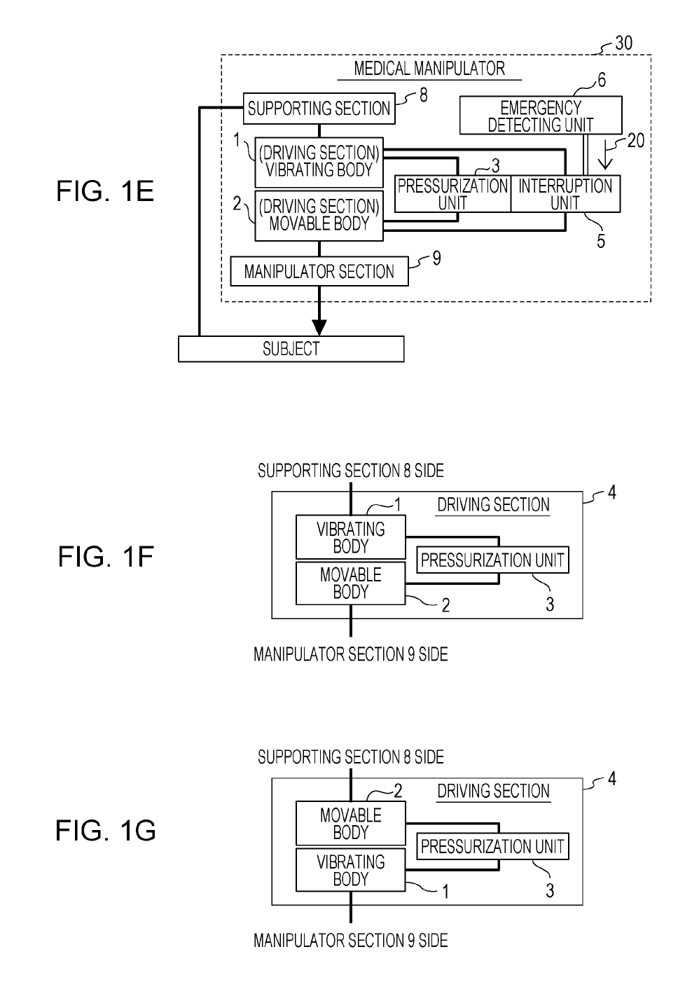

Referring next to FIGS. 1A to 1G and FIG. 9, a first aspect of the present invention will be described. FIGS. 1A to 1D show, in areas enclosed by the dotted lines, the connection relationship among the components of a medical manipulator 30 including a vibration-type actuator according to the first aspect of the present invention. FIGS. 1F and 1G show the connection relationship among the components of the driving section 4 shown in FIGS. 1A to 1D. FIG. 9 is a schematic cross-sectional view showing, in outline, the configuration of a ring-shaped ultrasonic motor including a piezoelectric device, applicable to the first aspect of the present invention.

As shown in FIGS. 1A to 1D, the medical manipulator 30 according to the first aspect of the present invention includes the supporting section 8, the driving section 4, the manipulator section 9, the emergency detecting unit 6, and the interruption unit 5. As shown in FIGS. 1F and 1G, the driving section 4 includes the vibrating body 1, the movable body 2, and the pressurization unit 3.

As shown in FIGS. 1A to 1D, the supporting section 8, the driving section 4, and the manipulator section 9 are disposed in this order.

As shown in FIGS. 1F and 1G, the supporting section 8 includes a portion fixed to one of the vibrating body 1 and the movable body 2 of the driving section 4, and the manipulator section 9 includes a portion fixed to the other of the vibrating body 1 and the movable body 2 of the driving section 4. The vibrating body 1 and the movable body 2 are brought into contact with each other by the pressurization unit 3 to generate a frictional force. With the above connection configuration, the medical manipulator 30 according to an embodiment of the present invention converts the relative movement of the vibrating body 1 and the movable body 2 to the relative movement of the supporting section 8 and the manipulator section 9 as the driving section 4 operates to achieve a manipulation operation. Such a connection configuration allows the relative distance between the manipulator section 9 and the subject to be controlled.

Next, the features and advantages of the medical manipulator 30 according to the first aspect of the present invention will be described using FIGS. 1A to 1G. As shown in FIGS. 1A to 1D, the first aspect of the present invention includes the interruption unit 5 at a portion of the path from the supporting section 8 via the driving section 4 to the manipulator section 9. The interruption unit 5 is capable of switching the connection from a connected state to a disconnected state on the basis of an emergency detection signal 20 transmitted from the emergency detecting unit 6. In the first aspect, providing the emergency detecting unit 6 and the interruption unit 5 causes a frictional force between the emergency detecting unit 6 and the interruption unit 5. In other words, this allows the interruption unit 5 to instantly release the connection between the supporting section 8 and the manipulator section 9 on the basis of the emergency detection signal 20 transmitted from the emergency detecting unit 6 also when a frictional force is generated between the vibrating body 1 and the movable body 2, that is, in a pressurized state.

“Connection” described above includes various mechanical connections and includes a connection via another structural member. Examples of another structural member include the interruption unit 5, which is one of the features of the first aspect of the present invention. Examples of the interruption unit 5 include various devices, such as a gas pressure cylinder including a pneumatic cylinder, a liquid-pressure cylinder including a hydraulic cylinder, an electromagnetic clutch, a mechanical clutch, and an air clutch provided that an interruption operation is executed in response to the emergency detection signal 20 from the emergency detecting unit 6.

The emergency detection signal 20 may be selected as appropriate from compressed air, compressed nitrogen, incompressible fluid, such as oil, water, and ethylene glycol, an electrical signal, and a mechanical transmission depending on the device of the interruption unit 5. If the interruption unit 5 includes an electromagnetic clutch as a component, an electrical signal or a pneumatic signal that uses the flow rate or pressure of compressed air as a parameter can be used as the emergency detection signal 20.

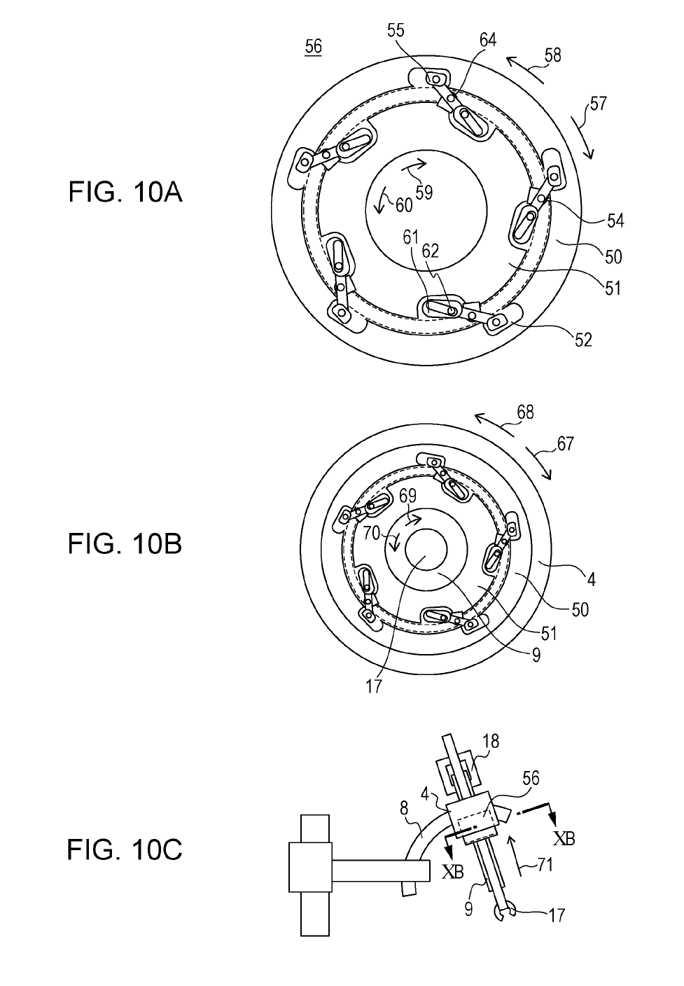

If the interruption unit 5 is constituted by a mechanical clutch, a one-way clutch 56 shown in FIG. 10A can be applied. The one-way clutch 56 includes an outer race 50, an inner race 51, inner race arms 54, sprags 55, pockets 52, counter weights 61, movable weights 62, fulcrums 64, and springs (not shown). One end of each spring is connected to an inner wall close to the fulcrum 64 in an accommodation space provided in the counter weight 61, and the other end is connected to the movable weight 62. The length of the interior of the pocket 52, the length of the opening of the pocket 52, and the length of the sprag 55 decrease in this order in the rotating direction. In other words, the pockets 52 each retain the sprag 55 therein under predetermined conditions to define cam surfaces that connect the inner race 51 and the outer race 50 together.

In the thus-connected one-way clutch 56, the connection between the inner race 51 and the outer race 50 is released by the following mechanism when the rotational acceleration of the inner race 51 in a counterclockwise direction 70 has exceeded a predetermined acceleration:
The rotation of the inner race 51 in the counterclockwise direction 70 increases.
An inertia force exerted on the movable weights 62 overcomes the elastic force of the springs to move the movable weights 62 in a direction away from the fulcrums 64.
The rotation moment M1 of the total weights of the counter weights 61 and the movable weights 62 applied to the fulcrums 64 increase.
The rotation moment M1 of the total weights of the counter weights 61 and the movable weights 62 applied to the fulcrums 64 becomes larger than the rotation moment M2 of the sprags 55 to the fulcrums 64.
The counter weights 61 move toward the outer race 50 due to a centrifugal force.
The sprags 55 come out of the pockets 52 disposed in the inner circumferential surface of the outer race 50 toward the inner race 51.
The connection between the inner race 51 and the outer race 50 is released.

The above descriptions indicated by “-” are made merely for ease of explanation of the operation mechanism of the one-way clutch 56 and do not necessarily show processes on the time axis; actually, the operations in the individual “-” occur substantially at the same time.

An acceleration condition ω₂(rad/s²) under which the connection between the inner race 51 and the outer race 50 is released can be changed as appropriate depending on the elastic constant and the length of the springs, the mass of the counter weights 61, the movable weights 62, and the sprags 55, the proportion of the lengths of the inner race arm 54, the sprag 55, the fulcrum 64, the counter weight 61, and the accommodating space in the rotating direction. In other words, in the case where the interruption unit 5 is the one-way clutch 56 shown in FIG. 10A, the unit constituted by <the inner race arm 54, the sprag 55, the pocket 52, the counter weight 61, the movable weight 62, the fulcrum 64, the spring (not shown), and the accommodating space> serves as the emergency detecting unit 6, and the emergency detection signal 20 is regarded as a difference ΔM between the rotation moments (=M2−M1).

Accordingly, the one-way clutch 56 shown in FIG. 10A can be regarded as serving as both the interruption unit 5 in FIG. 1B and the emergency detecting unit 6 that outputs the emergency detection signal 20 to the interruption unit 5.

When the inner race 51 rotates in a clockwise direction 59, the movable weights 62 stay at the fulcrum 64 side, and thus, an increase in the rotation moment of the counter weight 61 to the fulcrum 64 is suppressed, so that the rotation moment does not exceed the rotation moment M2 of the sprags 55 to the fulcrums 64, and thus, the sprags 55 are pushed against the outer race 50, staying in the pockets 52. As a result, the inner race 51 and the outer race 50 maintain the connected state.

Also in the feature of the vibration-type actuator, a normal manipulating acceleration ω₀(rad/s²) of the driving section 4 can be in a sufficiently lower acceleration range than the acceleration ω₁(rad/s²) of the manipulator section 9 by a manual operation in an emergency. By setting an acceleration condition ω₂(rad/s²) for releasing the connection between the manipulator section 9 and the driving section 4 by a manual operation in an emergency to satisfy ω₀≦ω₀ₘₐₓ<<ω₂≦ω₁, where the upper limit acceleration of the normal manipulating acceleration ω₀ is ω₀ₘₐₓ(rad/s²), both a manipulating operation in forward and reversal directions as a medical manipulator and an operation for releasing the connection of the interruption unit 5 in an emergency in a direction away from the subject can be achieved.

Accordingly, in the case where the one-way clutch 56 shown in FIG. 10A is used, the first aspect shown in FIG. 1B can be achieved by fixing the inner circumference of the inner race 51 to the outer circumference of the manipulator section 9 and fixing the outer circumference of the outer race 50 to the inner circumference of the driving section 4, as shown in FIG. 10B. With this configuration, when a predetermined external force 71 having a strength component directed in a direction away from the subject is exerted on the manipulator section 9, as shown in FIG. 1B, the manipulator section 9 in FIG. 10B rotates in the counterclockwise direction 70 under the condition that the acceleration is larger than the acceleration condition ω₂(rad/s²) for releasing the connection, so that the connection between the inner race 51 and the outer race 50 is released, and thus, the connection between the manipulator section 9 and the driving section 4 is released.

The interruption unit 5 according to the first aspect of the present invention shown in FIGS. 1A to 1G further includes a modification in which the one-way clutch 56 shown in FIG. 10A further includes an electromagnet (not shown) to constitute an electromagnetic clutch mechanism.

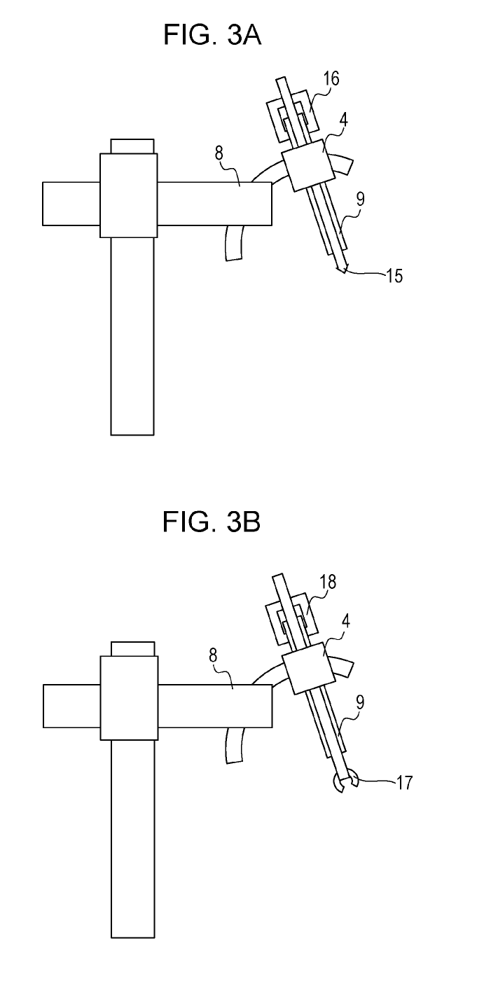

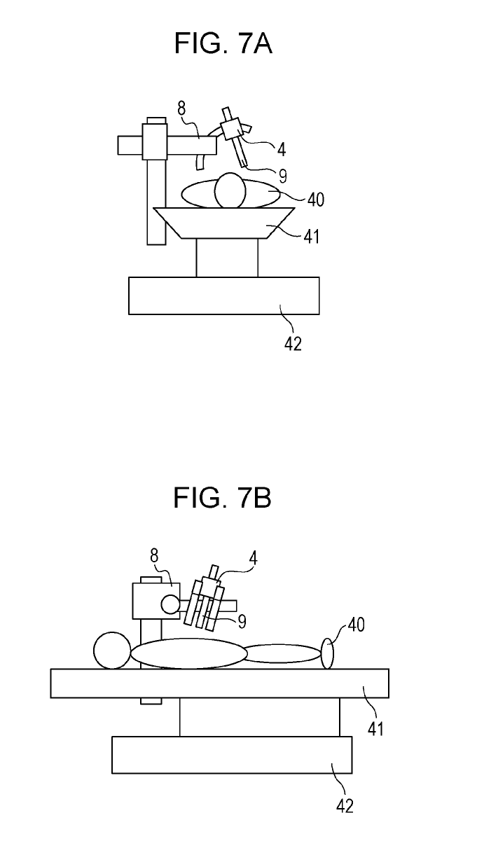

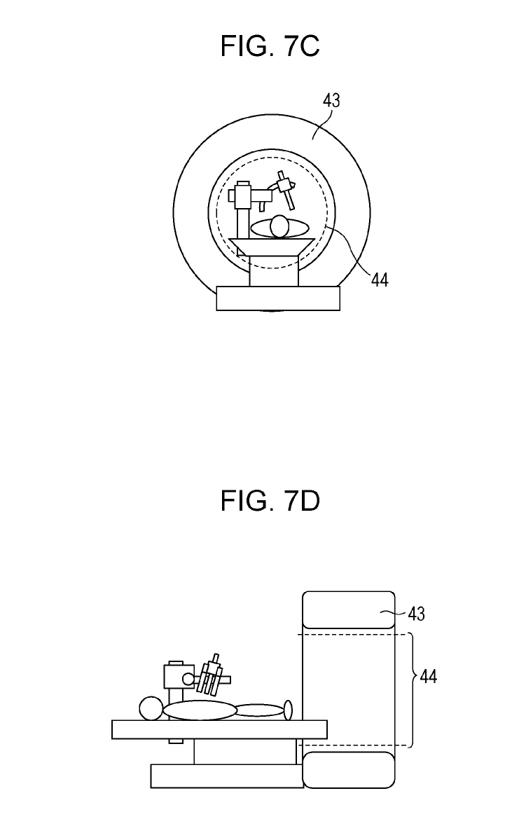

Referring next to FIGS. 3A and 3B and FIGS. 7A and 7B, the supporting section 8 will be described. FIGS. 7A and 7B show the positional relationship between the medical manipulator 30 according to an embodiment of the present invention and a subject 40 and the connection relationship between the medical manipulator 30 and a bed 41.

Positioning of the supporting section 8 to the subject 40 is defined relative to the surface of the subject 40, an outfit fixed to the subject 40, a jig placed on the clothes, part of an external unit, such as the bed 41, or the like. In FIGS. 7A and 7B, the supporting section 8 is fixed to the bed 41.

The supporting section 8 may have a light-weight, rigid structure so as to be capable of supporting the driving section 4 and the manipulator section 9 stably for the masses and operations thereof. Furthermore, the supporting section 8 may include an adjusting mechanism having flexibility in adjusting the rotation, the positions of straight lines, curves, etc., and the direction in view of flexibility in positioning the driving section 4 to the supporting section 8. In FIGS. 7A and 7B, the supporting section 8 has an adjusting mechanism that enables heightwise position adjustment, horizontal position adjustment, azimuth angle adjustment, and elevation angle adjustment. Furthermore, the above-described adjusting mechanism can be an adjusting mechanism capable of remote positioning with a predetermined driving source.

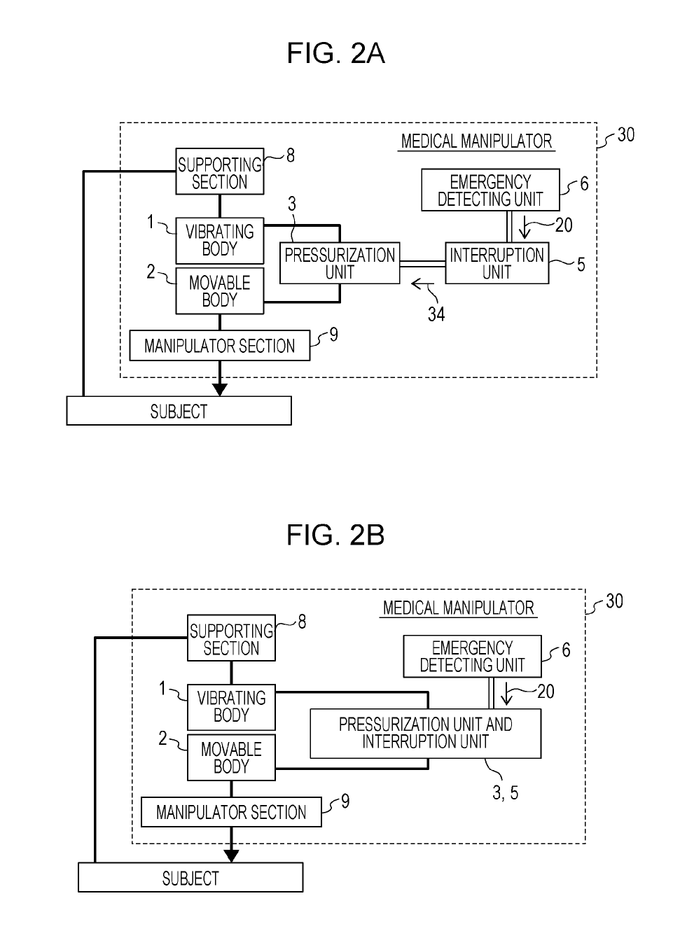

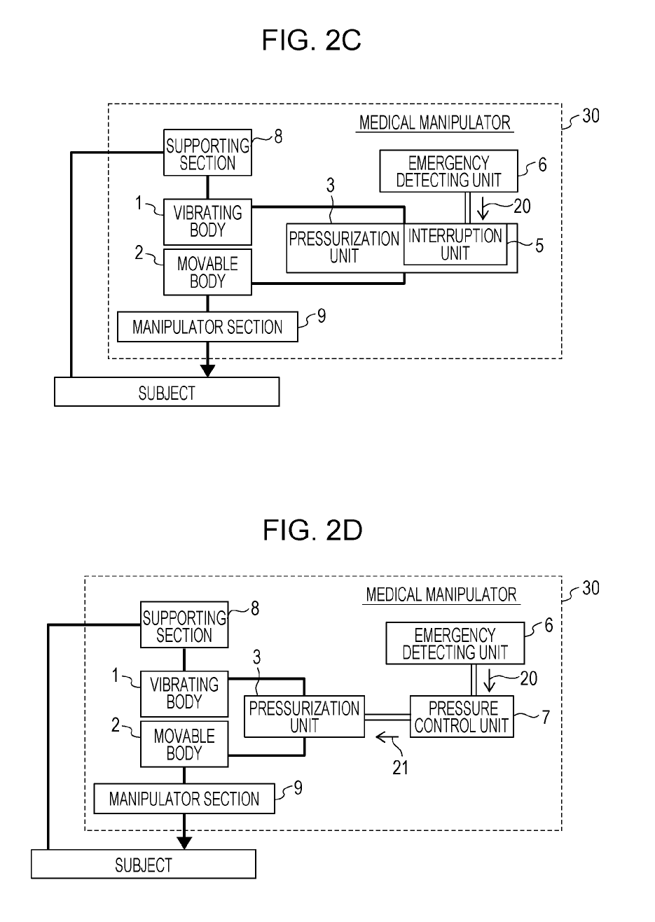

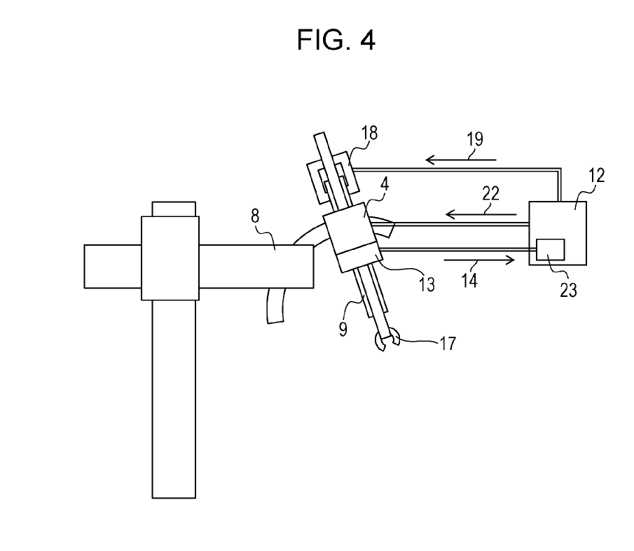

Referring next to FIG. 4, an example of the connection between a driving unit 12 that controls the medical manipulator 30 according to an embodiment of the present invention and the medical manipulator 30 will be described. FIG. 4 shows a medical-manipulator control system in which the driving unit 12 is connected to the driving section 4 of the medical manipulator 30 shown in the areas enclosed by the dotted lines in FIGS. 1A to 1E and FIGS. 2A to 2D. The manipulator section 9 is fixed to one end of the driving section 4, and the other end of the driving section 4 is fixed to the supporting section 8. This connection relationship allows the position of the manipulator section 9 relative to the supporting section 8 can be controlled. Furthermore, the medical manipulator 30 includes a positional-information detecting unit 13 and a receiver 23, as a configuration allowing more accurate position control, so that a positional-information signal 14 about the manipulator section 9 relative to the driving section 4 can be transmitted to the driving unit 12. FIG. 4 shows a configuration in which the receiver 23 that receives the positional-information signal 14 transmitted from the positional-information detecting unit 13 is provided at the driving unit 12. Furthermore, FIG. 4 shows that the driving unit 12 transmits a manipulator-unit driving signal 22 to the driving section 4 on the basis of the positional-information signal 14 received by the receiver 23.

Referring next to FIGS. 3A and 3B, an example in which an additional function is added to the medical manipulator 30. FIGS. 3A and 3B show configurations in which the medical manipulator 30 shown in FIG. 4 is applied to a medical puncturing system A and a medical biopsy system B, respectively. FIG. 3A shows a medical system in which a puncturing device 15 that punctures the body of the subject is connected relatively movably to the manipulator section 9. One end of the puncturing device 15 is fixed to a puncturing-device driving section 16, and the puncturing-device driving section 16 is fixed to the manipulator section 9. Such connection relationship allows position control of the puncturing device 15 relative to the manipulator section 9 and position control of the puncturing device 15 relative to the supporting section 8. FIG. 3B shows a biopsy system in which a collecting device 17 capable of collecting biological tissue of the subject and a collecting-device driving section 18 are disposed instead of the puncturing device 15 and the puncturing-device driving section 16 in FIG. 3A, respectively. As described above, any desired devices, such as a treatment support jig, an examination jig, and a sensor, may be connected to the manipulator section 9 of the medical manipulator 30 to provide higher performance and improve the fail-safe mechanism.

Although not shown in FIGS. 3A and 3B, the puncturing-device driving section 16 and the collecting-device driving section 18 are each connected to a driving circuit. The driving circuits for the additional functions may be integrated with the driving unit 12 shown in FIG. 4. The above integration allows the position control of the manipulator section 9 and the operations of the additional functions to be performed in a unified way. FIG. 4 shows that a collecting-device driving signal 19 is transmitted from the driving unit 12 to the collecting-device driving section 18.

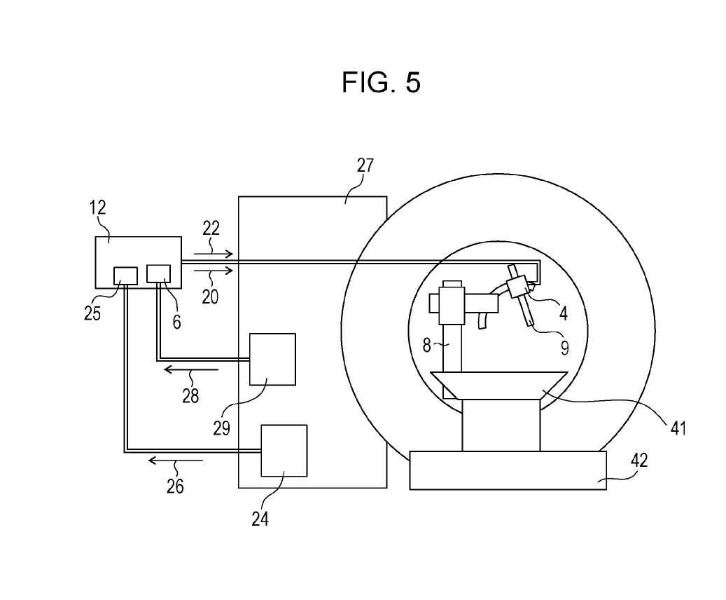

Referring next to FIG. 5, an example of connection between the emergency detecting unit 6 of the medical manipulator 30 according to an embodiment of the present invention and an external unit 27 will be described. FIG. 5 shows the connection relationship between the driving unit 12 of the present invention and the external unit 27 in the case where an MRI apparatus is applied as the external unit 27. The driving unit 12 includes the emergency detecting unit 6. The emergency detecting unit 6 is connected to an emergency-signal transmitting section 29 of the external unit 27. The emergency-signal transmitting section 29 transmits an emergency signal 28 to the emergency detecting unit 6 when the external unit 27 detects an emergency, such as vibration or a leak of electricity, or a manual instruction of the operator or the subject. Furthermore, the external unit 27 may include a transmitter 24 so as to transmit a target-position signal 26 to a target-position receiver 25 of the driving unit 12 on the basis of target-position information stored in the external unit 27. This configuration allows the driving unit 12 to have functions for comparing the positional information on the manipulator section 9 with the target position of the manipulator section 9, transmitting the normal manipulator-unit driving signal 22, and transmitting the emergency detection signal 20 as necessary. Emergencies included in the above emergency signal 28 transmitted from the external unit 27 include incorrect positions of the components of the medical manipulator 30 (the driving section 4, the supporting section 8, and the manipulator section 9), the bed 41, and a bed foundation 42 and emergencies, such as vibration.

The connection between the medical manipulator 30 according to an embodiment of the present invention and the subject 40 and the connection between the medical manipulator 30 and a medical imaging apparatus will be described using FIGS. 7A and 7B and FIGS. 7C and 7D, respectively. FIGS. 7A and 7B are schematic layout drawings of a medical system in which the medical manipulator 30 of the present invention is connected to a movable bed composed of the bed foundation 42 and the bed 41 as viewed from the lateral direction and the longitudinal direction of the bed 41 in a horizontal plane. In FIGS. 7A to 7D, the subject 40 can be restrained on the bed 41 with a positioning member, such as a belt and a cushion (not shown). Accordingly, the supporting section 8 can be substantially positioned to the subject 40 by fixing the supporting section 8 to the bed 41. FIGS. 7C and 7D are schematic layout drawings of a medical imaging apparatus (MRI apparatus 43) equipped with the movable bed shown in FIGS. 7A and 7B to which the medical manipulator 30 is applied.

The work distance, the size, and so on of the medical manipulator 30 according to an embodiment of the present invention are set so that image acquisition by the medical imaging apparatus is not hindered. In FIGS. 7C and 7D, the medical manipulator 30 is connected to the bed 41 so as to be accommodated in a bore 44 of the MRI apparatus 43.

Referring next to FIGS. 1A to 1G, FIGS. 2A to 2D, and FIGS. 6A and 6B, a second aspect of the present invention will be described. FIGS. 2A to 2D show a plurality of configurations corresponding to FIG. 1E. These configurations are referred to as the second aspect of the present invention. In the second aspect, the interruption unit 5 is disposed so as to directly interrupt the pressuring force exerted between the vibrating body 1 and the movable body 2, as shown in FIG. 1E and FIGS. 2A to 2C.

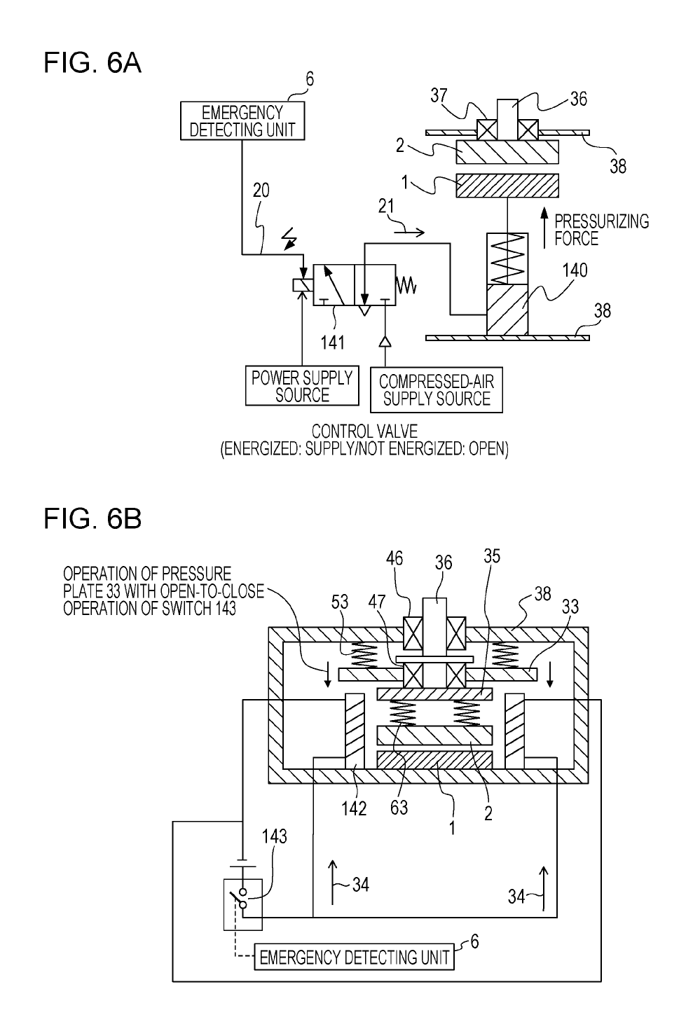

FIG. 2A shows a configuration in which the pressurization unit 3 and the interruption unit 5 are disposed in series. FIGS. 2B and 2C show cases where the pressurization unit 3 and the interruption unit 5 are integrated to one device having both the individual functions. A specific example thereof is shown as the schematic cross-sectional view in FIG. 6B. FIG. 2D shows a configuration in which the pressurization unit 3 and the pressure control unit 7 are disposed in series. The configuration in FIG. 2D allows the pressure control unit 7 connected in series to the pressurization unit 3 to control the pressurizing force. A specific example of the configuration shown in FIG. 2D is shown in the schematic cross-sectional view of FIG. 6A. The operations in FIGS. 6A and 6B will be described using examples.

A feature of the second aspect is a structure for interrupting the static torque itself of the vibration-type actuator in an emergency because of the configurations shown in FIGS. 2A to 2D. In the second aspect, the interruption unit 5 can be accommodated in the device structure of the vibration-type actuator, which is advantageous particularly in reducing the number of parts, size, and weight. With the configuration in which the interruption unit 5 is provided outside the driving section 4, the mechanical accuracy of the interruption unit 5 influences the position control accuracy of the medical manipulator. However, in the second aspect, the medical manipulator 30 is not substantially influenced by backlash, thus allowing the manipulator protection performance to be enhanced without reducing the position control accuracy of the medical manipulator 30 in normal operation.

In both the first and second aspects of the present invention, at least one of the pressurization unit 3 and the interruption unit 5 is selected for a predetermined period to perform the pressurizing operation or the interrupting operation. In the first and second aspects, the interruption unit 5 is always operated, and the pressurization unit 3 is selected for operation for a predetermined period to constitute a normally-off pressurizing mechanism.

Examples of the pressurization unit 3 include various devices, such as a reversible deformable member, such as an elastic member, a gas pressure cylinder including a pneumatic cylinder, a liquid pressure cylinder including a hydraulic cylinder, an electromagnetic clutch, and a mechanical clutch. In constituting the above-described normally-off pressurizing mechanism, a device having an affinity for a remote controller for the gas pressure cylinder, the liquid pressure cylinder, or the electromagnetic clutch may be applied.

Since the medical manipulator 30 according to an embodiment of the present invention includes a vibration-type actuator, it is possible to provide a medical system equipped with a fail-safe mechanism that can protect the medical manipulator 30 in the event of an emergency while maintaining the feature of high-accuracy direct drive as an advantage of eliminating the need for a reduction gear.

Furthermore, since the medical manipulator 30 has a fail-safe mechanism that operates in response to an emergency detection signal output from an external unit, a lock system using the positional information of the external unit can be achieved, thus further ensuring protection of the medical manipulator 30.

Examples of the external unit include medical imaging apparatuses, such as an MRI apparatus, a radiation imaging apparatus, and an ultrasonic imaging apparatus.

Applications of the medical manipulator 30 according to an embodiment of the present invention include biopsy, a surgery assistant, a higher cerebral function test using functional MRI analysis, and rehabilitation.

### First Embodiment

In this embodiment, the medical manipulator 30 according to the second aspect of the present invention, shown in FIG. 2D, is configured using the vibration-type actuator including the pressure control unit 7 as an interruption unit, shown in FIG. 6A, and the biopsy system shown in FIG. 4 is configured.

First, the biopsy system of the first embodiment will be described.

FIG. 4 is a diagram that schematically shows a configuration in which a medical manipulator of the first embodiment is applied to a biopsy system. The driving unit 12 includes a control circuit (not shown) for controlling the driving section 4. The biopsy system of the first embodiment further includes the positional-information detecting unit 13 connected to the manipulator section 9. The positional-information detecting unit 13 is electrically connected to the driving unit 12 to be capable of feedback control of the driving section 4 on the basis of the positional information on the manipulator section 9. The manipulator section 9 is connected to the collecting device 17 via the collecting-device driving section 18 to constitute a biopsy system.

FIG. 6A schematically shows the connection relationship among the vibration-type actuator applied to the first embodiment, the pressure control unit 7, and the emergency detecting unit 6. The relationship among the components and the operations thereof will be described using FIG. 6A. The vibration-type actuator of the first embodiment is configured such that the movable body 2 connected to the rotation shaft 36 is rotatably connected to the housing 38 via the bearing 37. Furthermore, the vibrating body 1 is connected to the housing 38 via a pneumatic cylinder 140 and is movable in the axial direction of the rotation shaft 36.

The pneumatic cylinder 140 is connected to a solenoid valve 141 through a plastic pipe so that the pressurizing force between the movable body 2 and the vibrating body 1 is remotely controlled depending on the supply level of compressed air from the solenoid valve 141 (hereinafter referred to as “pneumatic level”). The solenoid valve 141 is a solenoid valve 141 for pneumatic control. The connection relationship in FIG. 6A will be paraphrased using FIG. 2D; the pressure control unit 7 equipped with the solenoid valve 141 is connected to the pressurization unit 3 equipped with the pneumatic cylinder 140 so as to be capable of controlling the pressurization unit 3 in accordance with the pressure control signal 21 using the pneumatic level as a parameter.

Since compressed air is released into the atmosphere while the control solenoid of the solenoid valve 141 is OFF (not energized), no pressurizing force is exerted on the vibration-type actuator by the pneumatic cylinder 140, thus releasing the pressure. In the first embodiment, this is defined as that the solenoid valve 141 outputs the pressure control signal 21 at low level.

Next, the solenoid valve 141 of the first embodiment will be described using FIG. 6A. The solenoid valve 141 receives the emergency detection signal 20 from the emergency detecting unit 6.

The emergency detecting unit 6 connects to a unit with which a manual instruction from an external unit (not shown), the operator, or the subject 40 can be input. The solenoid valve 141 also connects to a power supply source and has an input device for compressed air supplied from a compressed-air supply source, such as a medical air supply system. The operating state of the solenoid valve 141 changes also depending on the pressure level of the compressed air. Specifically, if it is determined using a predetermined threshold value that compressed air sufficient for the pneumatic cylinder 140 to generate a pressurizing force is not obtained, the solenoid valve 141 comes in the same state as that when the solenoid is OFF.

When the emergency detecting unit 6 detects an emergency, the emergency detecting unit 6 outputs the negative logic emergency detection signal 20 to the pressure control unit 7. The negative logic allows the emergency detection signal 20 to serve as an interlock signal for the solenoid of the solenoid valve 141, thus allowing a fail-save mechanism that interrupts the power to the solenoid valve 141 in an emergency to be constructed.

The solenoid valve 141 of the first embodiment is regarded as receiving input of three different signals, that is, the emergency detection signal 20 output from the emergency detecting unit 6, a pneumatic signal output from the compressed-air supply source, and a supply power voltage signal output from the power supply source. In addition, an output valve to the cylinder and a release valve to the atmosphere described above are provided. Thus, the solenoid valve 141 of the first embodiment is regarded as outputting the pressure control signal 21 at low level provided that at least one of the three inputs described above is at low level. Thus, the pressurizing mechanism of the first embodiment constituted by the pressurization unit 3 and the pressure control unit 7 is considered to be a normally-off type (normally depressurized type).

As describe above, both when one of power supply and compressed air supply to the medical manipulator 30 of the first embodiment is interrupted and when the emergency detection signal 20 is detected, the solenoid valve 141 serving as the pressure control unit 7 outputs the pressure control signal 21 at low level.

As a result, the interval between the vibrating body 1 and the movable body 2 is depressurized to bring the vibrating body 1 and the movable body 2 into a rotatable state.

The solenoid valve 141 and the pneumatic cylinder 140 used in the first embodiment can be replaced with another pressure control unit 7 and another pressurization unit 3. For example, the pneumatic cylinder 140 can be replaced with another gas pressure control type that uses dry nitrogen gas or the like as a medium. The configuration of the first embodiment uses “the pressure or flow rate of compressed air” as a medium of the pressure control signal 21. In the present invention, the pressure control signal 21 may be “an electrical signal” or “the pressure or flow rate of liquid” by using incompressible liquid. However, the use of compressed air as a medium of the pressure control signal 21 has the advantage of higher compatibility with an external unit than liquid pressure control using a hydraulic cylinder in terms of the fact that it is easy to use a nonmagnetic material for a member disposed in the vicinity of the pressurization unit 3. Furthermore, this has an advantage in that the medium (air) after the control can be released into the atmosphere without reflux, so that the system can be made compact, and there is no need to concern degradation of the medium.

Here, conditions for the emergency detection signal 20 to become active (low level) will be described using specific examples. The first example is a case where an emergency signal is received from an external unit. For example, in an MR-guided surgery system in which the medical manipulator 30 cooperates with an MRI, if the MRI determines that an emergency has occurred, a message noticing the emergency is given to the medical manipulator 30 by communication with each other. Then, the medical manipulator 30 can shift the vibration-type actuator to a depressurized state. The second example is a case where an acceleration sensor is provided as the emergency detecting unit 6, with which a predetermined magnitude of vibration or more is detected. If it is determined using an appropriate threshold value that an earthquake has occurred, the vibration-type actuator is shifted to a depressurized state, so that the subject 40 can be given an appropriate treatment, and the medical manipulator 30 can be protected. The third example is a case where power supplied to the system is interrupted due to a power failure or an accident. In this case, the emergency detection signal 20 that serves also as a control signal for the solenoid valve 141 is a negative logic signal, so that the medical manipulator 30 automatically shifts to a depressurized state, and thus the medical manipulator 30 can be protected. The fourth example is a case where a manual instruction from the doctor, the operator, or the subject 40 is given. Providing an emergency switch serving as the emergency detecting unit 16 allows the medical system to be manually shifted to a depressurized state when a human detects an emergency due to a system failure or the like. Although the four cases have been described above, the present invention is not limited thereto. It is possible that the present invention has a configuration in which when an abnormality in the pressure of the setup location due to, for example, submergence, is detected by a pressure sensor, the pressure is released; a configuration in which an emergency can be reasonably detected; or a configuration in which a plurality of emergencies can be coped with by the logical OR thereof.

Although the solenoid valve 141 according to an embodiment of the present invention is a solenoid valve that controls an output pneumatic level relative to an input pneumatic level, an interruption unit applicable to the present invention is not limited thereto. For example, the present invention also includes a connection configuration in which a pressure sensor (not shown) is provided at the input side of the solenoid valve 141, and the output of the above pressure sensor is input to the emergency detecting unit 6.

### Second Embodiment

In this embodiment, the medical manipulator 30 according to the second aspect of the present invention, shown in FIG. 2B, is configured using the vibration-type actuator equipped with the interruption unit, shown in FIG. 6B, and the biopsy system shown in FIG. 4 is configured.

FIG. 6B is a schematic diagram showing, in outline, the configuration of the vibration-type actuator and the pressurizing mechanism applied to the second embodiment.

In the second embodiment, the pressurizing mechanism for the vibrating body 1 and the movable body 2 described in the first embodiment is changed.

In the second embodiment, the configurations of the vibration-type actuator and the emergency detecting unit 6 are the same as those in the first embodiment and are examples of the second aspect of the present invention as in the first embodiment.

The correspondence relationship between the reference signs of the vibration-type actuator applied to the second embodiment shown in FIG. 6B and the reference signs of the medical manipulator 30 shown in FIG. 2B will be shown below.

In the second embodiment, the pressurization unit 3 shown in FIG. 2B includes a pressure plate 33, an electromagnet 142, a movable-body-side elastic body 63, the rotation shaft 36, and a fixed bearing 47. The interruption unit 4 shown in FIG. 2B includes a switch 143, a housing-side elastic body 53, the pressure plate 33, and the rotation shaft 36. The emergency detecting unit 6 shown in FIG. 2B corresponds to the switch 143. A depressurization signal 34 shown in FIG. 2B is a direct current (depressurization signal) 34 flowing in the electromagnet 142 as the switch 143 closes.

Next, the operation of the pressurizing mechanism that controls the vibration-type actuator of the second embodiment will be described.

The vibrating body 1 is fixed to one end of the housing 38. The movable body 2 is fixed to the other end of the housing 38 via the movable-body-side elastic body 63, the rotation shaft 36, a movable bearing 46, the fixed bearing 47, the pressure plate 33, and the housing-side elastic body 53. The length and the elastic constant of the housing-side elastic body 53 are set so that, when the electromagnet 142 is OFF (not energized), the housing-side elastic body 53 is not elastically deformed, and the metallic pressure plate 33 is separated from the electromagnet 142, and when the electromagnet 142 is turned ON (energized), the metallic pressure plate 33 comes into contact with the electromagnet 142. The length and the elastic constant of the movable-body-side elastic body 63 are set so that, when the electromagnet 142 is OFF, the movable-body-side elastic body 63 is not elastically deformed, and the movable body 2 is separated from the vibrating body 1, and when the electromagnet 142 is turned ON, the movable body 2 and the vibrating body 1 come into contact with each other and come into a specific pressurized state. Hence, the second embodiment having the movable bearing 46 is configured to allow the vibrating body 1 and the movable body 2 to be separated from each other and to come into contact with each other; however, the present invention is not limited thereto provided that the same function can be achieved.

Providing the pressurization unit 3 and the interruption unit 5 that satisfy such connection relationship allows the vibration-type actuator of the second embodiment to achieve the normally-off type (normally depressurized) pressurizing mechanism, as in the first embodiment.

The second embodiment achieves a medical manipulator as an example of the second aspect of the present invention and includes a fail-safe mechanism having the same function as that of the first embodiment.

Although the second embodiment is configured such that the housing-side elastic body 53 is disposed as a reverse spring, the present invention is not limited thereto. For example, a configuration in which the housing-side elastic body 53 is omitted from the vibration-type actuator shown in FIG. 6B is also the second aspect of the present invention. Also in this case, the pressurizing force between the vibrating body 1 and the movable body 2 when the electromagnet 142 is OFF is completely released, although the distance therebetween is not specified, so that the movable body 2 can rotate relative to the vibrating body 1.

### Third Embodiment

This embodiment is an example in which the medical manipulator 30 shown in FIG. 1B according to the first aspect of the present invention is configured using the one-way clutch 56 shown FIG. 10A, and the biopsy system shown in FIGS. 10B and 10C is configured.

The biopsy system of the third embodiment shown in FIG. 10C is a modification of the biopsy system shown in FIG. 3B. FIG. 10C shows a configuration in which the one-way clutch 56 shown in FIG. 10A is connected, as the interruption unit 5 in FIG. 1B, between the manipulator section 9 and the driving section 4 of the biopsy system shown in FIG. 3B. FIG. 10B is a schematic cross-sectional view of the connected portion of the driving section 4—the interruption unit 5—the manipulator section 9 of the biopsy system shown in FIG. 10C, taken along line XB-XB.

In the third embodiment, when an external force is exerted on the manipulator section 9 in a direction 71 in which the manipulator section 9 is moved away from the subject 40 in FIG. 10C, the manipulator section 9 shown in FIG. 10B rotates in the counterclockwise direction 70 together with the collecting device 17. When the rotation in the counterclockwise direction 70 exceeds a predetermined acceleration, the connection between the inner race 51 and the outer race 50 of the one-way clutch 56 shown in FIG. 10A is released by the cam mechanism constituted by the inner race arm 54, the sprag 55, the pocket 52, the counter weight 61, the movable weight 62, the fulcrum 64, the space accommodating the movable weight 62, and the spring (not shown) that connects the movable weight 62 and the inner race arm 54. As a result, the connection between the manipulator section 9 fixed to the inner race 51 and the driving section 4 fixed to the outer race 50 is released.

As described above, in any medical manipulators described in the first to third embodiments, the connection between the supporting section 8 and the manipulator section 9 is released in an emergency, thereby reducing the possibility of damaging the medical manipulator 30.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-135452, filed Jun. 15, 2012, which is hereby incorporated by reference herein in its entirety.

1. A medical manipulator comprising:
a supporting section;
a driving section supported by the supporting section and including a vibrating body in which vibration waves are excited by application of an alternating voltage, a movable body that moves relative to the vibrating body by receiving the vibration waves, and a pressurization unit configured to exert pressure between the vibrating body and the movable body;
a manipulator section supported by the driving section;
at least one emergency detecting unit configured to detect an emergency; and
an interruption unit configured to interrupt transmission of torque between the manipulator section and the supporting section on the basis of an emergency detection signal transmitted from the emergency detecting unit.

2. The medical manipulator according to claim 1, wherein the interruption unit is one of an electromagnetic clutch and a mechanical clutch connected somewhere between the supporting section, the driving section, and the manipulator section.

3. The medical manipulator according to claim 2, wherein the mechanical clutch is a one-way clutch that comes into an interrupted state when a predetermined external force having a strength component in a direction away from a subject is exerted on the manipulator section or the driving section.

4. The medical manipulator according to claim 2, wherein the mechanical clutch is one of an air clutch, a hydraulic clutch, and a brake system having an external input section and coming into an interrupted state when input to the external input section exceeds a predetermined level.

5. The medical manipulator according to claim 1, wherein the interruption unit is connected to the pressurization unit.

6. The medical manipulator according to claim 5, wherein the interruption unit is a pressure control unit configured to control the pressure.

7. The medical manipulator according to claim 5, wherein the pressurization unit includes one of a gas pressure cylinder and a liquid pressure cylinder.

8. The medical manipulator according to claim 5, wherein the pressurization unit is an electromagnetic clutch including an electromagnet and an elastic body.

9. The medical manipulator according to claim 6, wherein the pressure control unit includes one of a gas-pressure control unit and a liquid-pressure control unit.

10. The medical manipulator according to claim 7, wherein the emergency detecting unit makes the emergency detection signal active when receiving an emergency signal transmitted from an external unit.

11. The medical manipulator according to claim 1, wherein the interruption unit is connected to the plurality of emergency detecting units and operates using logical OR in response to a plurality of emergency detection signals transmitted from the plurality of emergency detecting units.

12. The medical manipulator according to claim 1, wherein the emergency detecting unit is configured to transmit an emergency detection signal that goes to low level in an emergency to the interruption unit.

13. The medical manipulator according to claim 12, wherein when detecting a vibration of a predetermined magnitude or greater, the emergency detecting unit transmits the emergency detection signal.

14. The medical manipulator according to claim 12, wherein when a manual instruction is given by a human, the emergency detecting unit transmits the emergency detection signal.

15. The medical manipulator according to claim 6, wherein when the pressure control unit receives the emergency detection signal, a pressure control signal that the pressure control unit outputs goes to low level.

16. The medical manipulator according to claim 15, wherein
the pressure control unit is connected to the plurality of emergency detecting units; and
when the pressure control unit receives the emergency detection signal from at least one of the plurality of emergency detecting units, the pressure control unit outputs the pressure control signal to the pressurization unit at low level.

17. A medical puncturing system comprising:
the medical manipulator according to claim 1; and
a puncturing device that is connected to the manipulator section and that is configured to puncture the body of a subject.

18. A biopsy system comprising:
the medical manipulator according to claim 1; and
a collecting device that is connected to the manipulator section and that is configured to collect biological tissue of a subject.

19. A medical system comprising:
the medical manipulator according to claim 1;
a driving unit that drives the medical manipulator; and
a medical imaging apparatus including a transmitter that transmits positional information of a predetermined target site,
wherein the driving unit includes a target-position receiver that receives positional information of the predetermined target site.

20. The medical system according to claim 19, wherein the medical imaging apparatus is one of an MRI apparatus, a radiation imaging apparatus, and an ultrasonic imaging apparatus.
